(19) United States
(12) Patent Application Publication
(10) Pub. No.: US 2021/0379038 A1
(43) Pub. Date: Dec. 9, 2021

(54) PHARMACEUTICAL COMPOSITIONS FOR INTRAVENOUS INFUSION IN PLASTIC BAGS WITH REDUCED ADSORPTION ON BAG MEMBRANE

(71) Applicant: Good Health, LLC, Princeton, NJ (US)

(72) Inventor: Basavaraj Siddalingappa, Gujarat (IN)

(73) Assignee: Good Health, LLC, Princeton, NJ (US)

(21) Appl. No.: 17/339,415

(22) Filed: Jun. 4, 2021

(30) Foreign Application Priority Data
Jun. 5, 2020 (IN) 202041023669

(51) Int. Cl. A61K 31/4422; A61K 47/12; A61K 47/26; A61K 47/34; A61K 47/02; A61K 47/10; A61K 9/00; A61K 47/24; A61J 1/10

(52) U.S. Cl. CPC A61K 31/4422; A61K 47/12; A61K 47/26; A61K 47/34; A61J 1/10; A61K 47/10; A61K 9/0019; A61K 47/24; A61K 47/02

(57) ABSTRACT

Liquid nicardipine compositions suitable for parenteral administration having improved stability are disclosed. The compositions are aqueous solutions having a pH of at least about 4.8, include nicardipine or a pharmaceutically acceptable salt thereof, sodium benzoate, a surfactant, a buffer and optionally a tonicity adjusting agent such as sorbitol. The liquid nicardipine compositions are preferably stored in a pharmaceutically-acceptable bag-type container. An inert gas such as nitrogen can be included therein if desired.

### CROSS-REFERENCE TO RELATED APPLICATIONS

This application claims the benefit of priority from Indian Patent Application Serial No. 202041023669, filed 5 Jun. 2020, the disclosure of which is incorporated herein by reference in its entirety.

### FIELD OF INVENTION

This invention relates to stable compositions of drugs which have a greater affinity to plastic or polymer surfaces of intravenous infusion bags. The invention pertains to composition, packing material and packing process to present drug product in stable condition for long time periods.

### BACKGROUND OF INVENTION

Drugs for intravenous infusion are often packed in glass bottles, plastic bags made from hydrophilic or hydrophobic polymers. However, considering ease of packing, transport and handling, plastic bags are preferred over glass bottles. Plastic bags present a unique challenge of loss of potency of packed drugs due to adsorption of drugs on to the surface.

Nicardipine HCl comes as both a concentrated solution in a vial and a dilute ready to use, premixed solution in a bag. Concentrated solutions in vials can be quite stable but after dilution, the solutions must be used immediately. The premixed solutions in bags have a lower pH which may not offer great comfort to patients.

Other drugs such as Insulin and Fentanyl come as vial products. Dilution before use is required for low dose continuous infusion. These drugs cannot be packed in bags as ready to infuse solutions due to their inherent affinity for the membrane of plastic infusion bags. Simple compositions with appropriate packing conditions to present such drugs as ready to use premix solution in bags are very much needed.

U.S. Pat. No. 7,612,102B2 describes the preparation of dilute, ready to use, premixed solutions of Nicardipine packed in plastic bags with long term stability. The compositions of this invention have a pH of around 4.0.

U.S. Pat. No. 7,659,291B2 describes treatment of hypertension patients using Nicardipine compositions having a pH value between 3.6-4.7, packed in plastic bags. The patent also describes the use of buffers for pH adjustment and osmotic agents for maintaining osmolality close to blood.

U.S. Pat. No. 8,455,524B2 describes treatment of hypertension using Nicardipine solution having a pH between 3.6-4.7, packed in plastic bags. Tonicity adjusting agents and buffers in the formulations are mentioned.

U.S. Pat. No. 9,364,564B2 describes Nicardipine compositions containing buffer, tonicity adjusting agents, and compositions have a pH between 3.6-4.7.

U.S. Pat. No. 9,549,994B2 describes pharmaceutical compositions of Nicardipine for intravenous infusion containing sulfo alkylated β-cyclodextrin and having a pH between 4.5-5.5.

U.S. Pat. No. 5,164,405A describes pharmaceutical compositions comprising Nicardipine in buffered solutions having pH in the range of 3.0-4.4. Non-chloride tonicity adjusting agents such as sorbitol, dextrose, mannitol, glucose, polyethylene glycol and glycerol are included in the composition to achieve the isotonicity with blood. However, the compositions are in the glass vials/ampoules.

US20070112041A1 describes nicardipine hydrochloride injection compositions comprising sorbitol in amounts sufficient to stabilize the injection preparation and a physiologically and pharmaceutically acceptable buffer, in an amount effective to maintain the pH of the composition at from about 3.0 to about 4.5. However again, these formulations are packed in ampoules.

EP0162705B1 describes injectable nicardipine hydrochloride compositions comprising an aqueous nicardipine hydrochloride solution containing from 2 to 7 w/v % of polyhydric alcohol and having a pH of 2.5 to 5.5. The solutions are stored in ampoules.

USRE34618E describes allegedly stable, injectable compositions of nicardipine hydrochloride in ampoule form which comprise an aqueous nicardipine hydrochloride solution containing 0.04 to 0.6 W/V % nicardipine hydrochloride and 2 to 7 W/V % of a polyhydric alcohol and wherein the pH of the solution is from 2.5 to 5, and the percentage of nicardipine hydrochloride remaining in the solution after a 12 week storage period at 60° C. is between 69.24 percent and 74.39 percent.

US20120214761A1 describes compositions containing an inclusion complex of nicardipine or a pharmaceutically acceptable salt thereof and a sulfo alkylated β-cyclodextrin.

U.S. Pat. No. 4,880,823A discloses injectable composition of nicardipine hydrochloride in ampoule form comprising an aqueous nicardipine hydrochloride solution containing from 0.04 to 0.6 W/V % nicardipine hydrochloride and from 2 to 7 W/V % of a polyhydric alcohol. The pH of the solution is from 2.5 to 5, and the percentage of nicardipine hydrochloride remaining in the solution after a 12-week storage period at 60° C. is between 69.24 percent and 74.39 percent.

EP0324982B1 discloses an inclusion complex of nicardipine or its hydrochloride with beta-cyclodextrin, wherein the molar ratio of nicardipine or its hydrochloride and beta-cyclodextrin is 1:0.9-1.1.

U.S. Pat. No. 5,002,958A describes methods of treating hypertension which comprises administering to a subject requiring such treatment an antihypertensive effective amount of 4′-ethyl-2-methyl-3-piperidinopropiophenone or a pharmacologically acceptable salt thereof.

Most of above-mentioned patents describe either formulations of Nicardipine in the vials/ampoules or those in bags with pH 3.6-4.7. None of these inventions mention stable formulations of Nicardipine bags with pH>4.7. Hence the stable formulations of Nicardipine in IV infusion bags with pH range of 4.7-7.0 are essential.

If the pH of nicardipine is close to the pH of blood, it would offer greater comfort to patients during injection which lasts for longer time.

It was reported that Nicardipine has pH dependent stability in aqueous solutions. The stability of Nicardipine increases as pH increases. It displays greater stability after pH 4.0. In contrast, if solutions are stored in a plastic container, the potency of Nicardipine is adversely affected by adsorption on to the plastic surface. Chemical stability offered by increasing pH of solution is adversely impacted by adsorption on to plastic surfaces, if solutions are to be stored in plastic container.

To preserve greater stability and increased patient compliance offered by Nicardipine solutions prepared at higher pH, potency loss due to adsorption on to plastic bags needs to be negated through technical interventions.

### SUMMARY OF THE INVENTION

The invention relates to the liquid formulations packed in plastic infusion bags for such drugs which have a greater affinity for a plastic membrane.

This invention also describes treatment of patients in need of treatment with compositions of the invention. Such methods include administering a drug-containing composition as described herein to a patient in need of such drug, preferably by administering the composition without further reconstitution or transformation of the formulation in the infusion bag prior to administration.

The invention also relates to methods of making and methods of packing compositions of this invention.

### DETAILED DESCRIPTION OF THE INVENTION

The invention relates to stable, liquid pharmaceutical compositions comprising Nicardipine, water, a hydrotropic agent such as sodium benzoate, a surfactant, and a buffer. The compositions preferably have a pH of at least 4.8 and the compositions are preferably ready to use, i.e., without requiring further dilution.

The compositions of this inventions are preferably packed, i.e., filled in or dispensed from plastic infusion bags. The compositions can also include a tonicity adjusting agent such as sorbitol and/or NaCl, and optional ingredients such as a pH adjustor, a preservative, and other ancillary excipients which will be apparent to those of ordinary skill.

The drug-containing compositions are preferably stored in suitable plastic packaging. The volume of the plastic packaging is such as to allow for an amount of an inert gas such as nitrogen which is sufficient to extend the stability of the formulation to be included therein if desired. Plastic bags made from non-polar polymers such as PP INERTA, GALAXY®, EXCEL®, VISIT® and VAILO can be used for storing of Nicardipine solution of this invention. Without limitation; suitable bags can be made from materials such as polypropylene, polyvinylchloride, polyethylene and ethyl vinyl acetate or any other compatible polymeric material. In another aspect, suitable bags comprise a copolyester, polyethylene or polyolefin, etc. surface in contact with the liquid nicardipine composition. For purposes of the present invention, it will be understood by those of ordinary skill that the term “bag” indicates a pharmaceutically acceptable bag made of plastic and which is commonly employed in containing pharmaceutical formulations and used in the administration of pharmaceuticals to patients via intravenous administration.

A “stable” composition of the invention means a pharmaceutical composition having sufficient stability at room temperature conditions to have utility as a pharmaceutical product. Preferably, a “stable” composition of the invention has sufficient stability to allow storage at room temperature conditions, preferably between about 15° C. and about 30° C., more preferably about 20° C. to about 25° C., most preferably about 25° C., and between about 55% to about 65% RH (e.g., about 60% RH), for a reasonable period of time, e.g., the shelf-life of the product which can be as short as one month but is typically six months or longer, more preferably one year or two years. A “stable” composition of the invention also includes specific ranges of impurities as described herein. Preferably, a “stable” composition is one which has minimal degradation of the at least one drug, e.g., it retains at least about 90% of un-degraded active, preferably at least about 95%, after storage at about 15-30° C. for a 1 to 3 year period of time.

The invention also relates to stable, liquid formulations of pharmaceutical compositions comprising, consisting of, or consisting essentially of Nicardipine or a pharmaceutically acceptable salt, ester, or prodrug thereof. These Nicardipine compositions are stable at room temperature conditions (25° C./60% RH) for extended periods of time (e.g., stable for >6 months, preferably >1 year, more preferably >2 years, with impurities less than or equal to acceptable limits (e.g., total impurities <3%, preferably <2%, more preferably <1%, as determined by HPLC after storing formulation at 40° C./75% RH for about 3 months). Surprisingly, it was found that the aqueous, ready to use nicardipine compositions as described herein containing sodium benzoate, a surfactant and having a pH of at least 4.8 can be included in bags and are long term stable. Such nicardipine compositions dispensed in bags can further optionally contain an inert gas such as nitrogen therein. The inert gas may alternatively be argon or xenon or a mixture of these gases with nitrogen.

Preferably, the Nicardipine is in its native form or its pharmaceutically acceptable salt, ester, or prodrug thereof (e.g., Nicardipine HCl). The term “Nicardipine” includes Nicardipine or a pharmaceutically acceptable salt, ester, or prodrug thereof. Nicardipine may alternatively be in the form of a complex. The Nicardipine may be present in the compositions of the invention in any amount, such as an amount ranging from about 0.015 mg/mL to about 0.5 mg/mL, preferably about 0.05 to about 0.4 mg/mL, more preferably about 0.1 to about 0.3 mg/mL.

The compositions of this embodiment have pH value of at least 4.8. In some embodiments the pH is in the range of between 4.8-8.0, preferably, between 4.8-6.0, more preferably between 4.8-5.5, more preferably 5.0-5.5.

The compositions of the present invention also include a hydrotropic agent such as sodium benzoate in an amount of from about 0.2 to about 2 mg/ml, with amounts of from about 0.5 to about 1.5 mg/mL, in some preferred aspects of the invention and amounts of about lmg/ml in other preferred aspects of the invention. In alternative aspects, hydrotropic agents besides or in addition to sodium benzoate can be used in the amounts described.

In some embodiments, the amount of inert gas included in the bags holding the drug-containing formulations can even be an amount above that which is typically employed in the packaging of a pharmaceutical composition and can be an amount deemed to be excessive or otherwise above that considered to be sufficient for stability purposes. For example, in most instances, parenteral compositions are typically filled into containers with from about 5 to about 20% of the actual volume of container. In certain aspects of the invention, the bags can include about 60% of actual volume of container or more of inert gas per container with the solution of Nicardipine comprising about 40% of bag volume or less. Otherwise, the compositions of the invention can be in standard bag packing, with close to 100% of stated volume of bag being filled with the nicardipine compositions, and if desired, about 5-10% headspace volume which available is filled with an inert gas.

In those embodiments where an excess of the inert gas is used, infusion bags containing Nicardipine are filled with excess of an inert gas, which is preferably nitrogen. The amount of nitrogen in the bag can be from about 10% volume of the bag to about 70% volume of the bag. Alternatively, the amount of nitrogen present in the bag can be between 20 to 70% of the volume of the bag, or 30 to 60% of the volume of the bag, or 40 to 60% of the volume of the bag. For example, if the volume of the inert gas is 50% in the bag, implies that plastic bag of 200 mL capacity would hold about 100 mL of the nicardipine solution and rest of the volume of the bags would be occupied by inert gas.

Per some embodiments, the compositions of the invention also contain at least one surfactant, at concentration of about 0.05 mg/mL to 5 mg/mL, more preferably about 0.1 to 3 mg/mL, most preferably about 0.5-2 mg/mL or about 0.6 to 1 mg/mL. Surfactants included in the compositions for Nicardipine may include but not limited to tweens, spans, polysorbates, poloxamers, cremophores, phospholipids such as phosphatidyl choline, PEGylated phospholipids such as 1, 2-Distearoyl-sn-glycero-3-phosphoethanolamine-Poly(ethylene glycol) (DSPE-PEG), e.g. DSPE-PEG2000, and other PEGylated compounds such as Cholesterol-PEG, Vitamin E-PEG etc. In other aspects, the surfactants can be fatty acid esters, PEG-fatty acids, PG-Phospholipids, etc. DSPE-PEG is a preferred surfactant.

The compositions of Nicardipine may also contain one or more buffering agents. Buffering agents include but are not limited to sodium citrate, sodium acetate, sodium phosphate at concentration of 0.05 mg/mL to 5 mg/mL, more preferably 0.1 to 3 mg/mL and most preferably 0.1 to 0.5 mg/mL.

Nicardipine compositions of this embodiment, may also contain excipients for tonicity adjustment. Tonicity adjusting agents belong to sodium chloride, glycerol, propylene glycol, dextrose, lactose, mannitol, sorbitol, sucrose. Suitable amounts may be from about 0.1 mg/mL to about 60 mg/mL, more preferably from about 0.6 to about 25 mg/mL, most preferably from about 0.8 to about 10 mg/mL. Sorbitol is one preferred tonicity adjusting agent.

Compositions of the embodiments may also contain preservatives, antioxidants, chelating agents.

### EXAMPLES

Materials and Methods

Materials:

Nicardipine HCl (>99% purity) was procured from TCI Chemicals (India) Pvt. Ltd. Phosphatidyl Choline (Soya) and 1, 2-Distearoyl-sn-glycero-3-phosphoethanolamine-Poly(ethylene glycol) (DSPE-PEG₂₀₀₀) were procured from Lipoid GMBH, Germany. Propylene Glycol AR grade was procured from Merck Lifesciences India Pvt Ltd. Polypropylene infusion bags (Inerta) were procured from Technoflex, Germany. Ail other chemicals and reagents were sourced locally were used without further purification.

Methods:

HPLC Method:

The method reported in USP for determination of assay of Nicardipine injection was used for assessing potency during stability testing of samples of Nicardipine. Method is as described below.

Preparation of Mobile Phase:

Buffer: 1.36 g/L of potassium dihydrogen phosphate in IL of water, To prepare mobile phase, 800 mL of methanol and 200 mL of buffer were mixed and filtered through 0.45μ filter.

Diluent: 500 mL of buffer was mixed with 500 mL of Methanol.

Preparation of Standard Solution:

The Nicardipine HCl, 10 mg was weighed into 100-mL volumetric flask. About 50 mL of diluent was added and sonicated to dissolve. Finally volume was made to 100 nit, with diluent.

Sample Preparation:

Sample either injected as is or diluted 1:1 with diluent.

Chromatographic Conditions:

Flow rate: 1 mL/min,

Column: Grace, 4.6-mm×250-mm; 5-μm packing L1.

Column temperature: 40° C.

Injection volume: 20 μL

Detection: 254 nm

### Example 1: Formulations with Soya Phosphatidyl Choline (Soya PC)

TABLE 1
Compositions with Soya PC

| Ingredients | Form-1 | | Form-2 | | Form-3 | |
|---|---|---|---|---|---|---|
| Nicardipine | 40 | mg | 40 | mg | 40 | mg |
| Soya PC | 20 | mg | 40 | mg | 30 | mg |
| Sodium | 200 | mg | 200 | mg | 200 | mg |
| Benzoate | | | | | | |
| Propylene | 4 | mL | 4 | mL | 4 | mL |
| Glycol | | | | | | |
| Water | 200 | mL | 200 | mL | 200 | mL |

Preparation:

Step 1: Sodium Benzoate was dissolved in water.

Step 2: Soya PC was dissolved in propylene glycol.

Step 3: Solutions from step-1 and step-2 were mixed.

Step 4: Nicardipine was added to step-3 and pH was adjusted 5.0 with dilute HCl solution and stirred until clear solutions was formed. Solution was filtered through 0.22μ nylon filter.

Packing:

About 50 mL of above formulations were filled into 200 mL polypropylene bags. Rest of the volume of bags were filled with Nitrogen. Bags were little bulged.

TABLE 2
Stability data for Form-1-3

| | % Assay | | | | | |
|---|---|---|---|---|---|---|
| | | 40 C./ | 40 C./ | 40 C./ | 40 C./ | 40 C./ |
| | | 75% | 75% | 75% | 75% | 75% |
| | | RH | RH | RH | RH | RH |
| Formulation | Initial | 1 Week | 15 Days | 1 month | 2 months | 3 months |
| Form-1 | 100.0 | 99.07 | 100.04 | 97.59 | 98.90 | 97.19 |
| Form-2 | 100.0 | 94.97 | 95.30 | 92.17 | 89.83 | 89.67 |
| Form-3 | 100.0 | 97.34 | 97.70 | 96.32 | 94.14 | 92.84 |

### Example 2: Formulations with and without Phospholipids

TABLE 3
Compositions for Form-4 to Form-8

| Ingredients | Form 4 | | Form 5 | | Form 6 | | Form 7 | | Form 8 | |
|---|---|---|---|---|---|---|---|---|---|---|
| Nicardipine HCl | 40 | mg | 40 | mg | 40 | mg | 40 | mg | 40 | mg |
| Sodium Chloride | 1.8 | gm | — | | 1.8 | gm | 1.8 | gm | — | |
| Propylene Glycol | — | | 4 | mL | — | | — | | 4 | mL |
| Sodium Benzoate | — | | 200 | mg | — | | 200 | mg | — | |
| Soya PC | — | | 20 | mg | — | | — | | 20 | mg |
| DSPE-PEG | — | | — | | 20 | mg | 20 | mg | — | |
| Distilled water | 200 | mL | 196 | mL | 200 | mL | 200 | mL | 196 | mL |

| | | | | | |
|---|---|---|---|---|---|
| pH | 5.04 | 5.01 | 5.08 | 5.05 | 5.02 |
| (Adjusted using | | | | | |
| dilute HCl) | | | | | |

Procedure:

Form-4: Sodium chloride was dissolved in water, Nicardipine was added to mixture and pH was adjusted to 5. Mixture was stirred till clear solution was formed.

Form-5: Same as Form-1-3.

Form-6: Sodium chloride and lipid were dissolved in water and Nicardipine was added to mixture and adjusted pH to 5 using dilute HCl. Stirred the mixture until it was cleared.

Form-7: Same as Form-6 but sodium benzoate was added to mixture along with sodium chloride and lipid.

Form-8: Same as Form-1-3 but without sodium benzoate.

All formulations were filtered through 0.22μ nylon filter.

Package:

25 mL of these formulations were stored in 100 mL polypropylene bags with rest of volume filled up with Nitrogen. Bags were little bit bulged.

TABLE 4
Stability data of Form-4 to Form-8

| | Test | Form-4 | Form-5 | Form-6 | Form-7 | Form-8 |
|---|---|---|---|---|---|---|
| S No | Parameter | Initial | Initial | Initial | Initial | Initial |
| 1 | Assay | 98.5 | 92.0 | 95.9 | 96.0 | 94.9 |

1M 40° C./75% RH

| | | | | | | |
|---|---|---|---|---|---|---|
| 2 | Assay vs STD | 84.6 | 90.5 | 93.0 | 93.0 | 96.0 |

2M 40° C./75% RH

| | | | | | | |
|---|---|---|---|---|---|---|
| 6 | Assay vs STD | 83.1 | 93.0 | 97.6 | 95.8 | 100.2 |

3M 40° C./75% RH

| | | | | | | |
|---|---|---|---|---|---|---|
| 19 | Assay vs STD | Not | 91.5 | 93.4 | 93.4 | 90.7 |
| | | analyzed | | | | |

Observations:
1. Results of Form-4 indicates that without surfactants and ingredients, i.e. just in plain buffer, the formulation does not offer great stability and potency decreases by about 17%, which is not acceptable. The decrease in potency is mainly due to adsorption as earlier reports suggest greater stability above pH 4.5 but adsorption thereafter.
2. Comparison of Form-5 and Form-8, indicates that to maintain the potency, not just phospholipids but also a hydrotropic agent such as sodium benzoate is needed. Form-5 retained potency even after three months (91 vs 92%), whereas Form-8, there close to 5% drop in potency vs initial.
3. Form-6 and Form-7, although data depicts comparable trend, but it was surprising that Form-6, without the hydrotropic agent, showed precipitation when stored at 25° C./60% RH. So, benzoate helps to keep the drug in solution and also prevent adsorption.
4. Overall, it is evident from the trend that both a surfactant and a hydrotropic agent such as sodium benzoate help to maintain stability of Nicardipine at about pH 5.0 when stored in plastic bags.

### Example 3: Formulation Packed with and without Nitrogen

TABLE 5
Composition with and
without nitrogen

| | Form-9 | |
|---|---|---|
| Ingredients | (Form-7 repeat) | |
| Nicardipine HCl | 40 | mg |
| Sodium Chloride | 1.8 | gm |
| Propylene Glycol | — | |
| Sodium Benzoate | 200 | mg |
| DSPE-PEG | 20 | mg |
| Distilled water | 200 | mL |

| | |
|---|---|
| pH | 5.02 |

Process: Same as Form-7.

TABLE 6
Stability data of Form-9

| | Stability time points | | | | |
|---|---|---|---|---|---|
| | | 1M 15 D | 1M 15 D | 3M | 3M |
| | | @40 C./75% RH | @40 C./75% RH | @40 C./75% RH | @40 C./75% RH |
| Parameters | Initial | (No nitrogen) | (Nitrogen) | (Nitrogen) | (No-Nitrogen) |
| Description | | Conforms | Conforms | Conforms | Conforms |
| pH | 5.02 | 5.25 | 5.28 | Not Analyzed | Not Analyzed |
| Osmolality | 293 | Not Analyzed | Not Analyzed | Not Analyzed | Not Analyzed |
| Assay | 102.33% | 92.13 | 95.05 | 96.00 | 93.71 |
| Nicardipine | Not analyzed | Not detected | Not detected | Not detected | Not detected |
| Monoacid^{a} | | | | | |
| Nicardipinepyridine | 0.13@RRT0.934 | 0.15@RRT0.929 | 0.18@RRT0.930 | 0.29@RRT0.930 | 0.24@RRT0.930 |
| analog^{b} | | | | | |
| Any unspecified | 0.05@RRT0.349 | 0.06@RRT0.347 | 0.05@RRT0.349 | 0.05@RRT0.23 | 0.05@RRT0.23 |
| impurities | 0.03@RRT0.360 | 0.08@RRT0.412 | 0.08@RRT0.412 | 0.12@RRT0.40 | 0.11@RRT0.40 |
| | 0.04@RRT0.412 | 0.17@RRT0.741 | 0.18@RRT0.743 | 0.34@RRT0.74 | 0.33@RRT0.74 |
| | 0.04@RRT0.843 | | | 0.15@RRT0.85 | 0.13@RRT0.85 |
| Total impurities | 0.29 | 0.46 | 0.49 | 0.95 | 0.86 |

All impurities are calculated as % area vs total area of all peaks.

Observation:
1. Form-9 stability results with and without nitrogen indicates that Nitrogen helps to maintain the potency of Nicardipine in the plastic bag.
2. However, it can be noted that pH raises to 5.28 after one month, so measures to control rise in pH needed.

### Example-4: Examples with Citric Acid and Sorbitol as pH Modifier and Stabilizer

TABLE 7
Composition for Form-10

| | Form-10 | |
|---|---|---|
| | QTY | QTY |
| INGREDIENTS | (mg/L) | (mg/mL) |
| Nicardipine HCl | 200 | 0.2 |
| DSPEPEG Sod. | 100 | 0.1 |
| Sodium Chloride | 7500 | 7.5 |
| Sodium Benzoate | 1000 | 1 |
| Sorbitol | 3840 | 3.84 |
| Citric Acid | 38.4 | 0.0384 |
| Purified water | Up to 1 L | Up to 1 mL |
| 5% Citric acid | Q.S to | Q.S to |
| | adjust pH | adjust pH |

Process: Same as Form-7.

TABLE 8
Stability data of Form-10

| | Form-10 at | | | |
|---|---|---|---|---|
| | 40° C./75% RH | | | |
| Test | Initial | 2 months | 3 months | 6 months |
| Description | Clear | Clear | Clear | Clear |
| | solution | solution | solution | Solution |
| pH at 25 ± 2° C. | 4.97 | 5.01 | 5.06 | 5.44 |
| Osmolality (mOsm/kg) | 291 | NT | NT | 295 |
| Assay (%) | 100.68 | 97.90 | 98.24 | 97.84 |
| Related substances (%) | % Impurity | | | |
| Nicardipine monoacid | NT | ND | ND | ND |
| (nicardipine related | | | | |
| compound A) | | | | |
| Nicardipine pyridine | 0.05 | 0.28 | 0.33 | 0.35 |
| analog (nicardipine | | | | |
| related compound B) | | | | |
| RRT 0.24 | 0.02 | 0.05 | 0.06 | ND |
| RRT 0.35 | ND | 0.10 | ND | ND |
| RRT 0.40 | 0.03 | ND | 0.07 | 0.12 |
| RRT 0.50 | ND | ND | ND | ND |
| RRT 0.58 | ND | ND | 0.04 | ND |
| RRT 0.74 | ND | 0.31 | 0.47 | 0.80 |
| RRT 0.84 | 0.04 | 0.10 | 0.19 | 0.55 |
| Total impurities | 0.14 | 0.74 | 1.16 | 1.82 |

ND: Not detected

Observation:
1. Form-10 pH remains almost unchanged even after 3 months at 40° C./75% RH, whereas Form-9 showed drastic increase in pH in same time period.
2. Assay remained same even after 6 months which could be due to containment of pH below 5.5.
3. Stability in terms of impurities, sorbitol has moderate positive effect.

Overall Summary
1. Results of Form-4 indicates that without surfactants and ingredients, just in plain buffer, the formulation does not offer great stability and potency decreases by about 17%, which is not acceptable. The decrease in potency is mainly due to adsorption as earlier reports suggest greater stability above pH 4.5 and but adsorption thereafter.
2. Comparison of Form-5 and Form-8, indicates that, to maintain the potency, not just phospholipids but also sodium benzoate is needed. Form-5 retained potency even after three months (91 vs 92%), whereas Form-8, there is close to 5% drop in potency vs initial.
3. Form-6 and Form-7, although data depicts comparable trend, it was surprising that Form-6, without benzoate, showed precipitation when stored at 25° C./60% RH.
4. Overall, it is evident from trend that both surfactant and sodium benzoate help to maintain stability of Nicardipine at about pH 5.0 when stored in plastic bags.
5. Form-9 stability results with and without nitrogen indicate that Nitrogen can help maintain the potency of Nicardipine in the plastic bag.
6. Form-10 pH remains almost unchanged even after 3 months at 40° C./75% RH, whereas Form-9 showed drastic increase in pH in same time period.
7. Assay remained nearly same even after 6 months; this could be due to containment of pH below 5.5.
8. Sorbitol has a positive effect on stability in terms of impurities.

1. A liquid nicardipine composition suitable for parenteral administration, comprising
a) nicardipine or a pharmaceutically acceptable salt thereof;
b) water;
c) a hydrotropic agent;
d) a surfactant; and optionally
e) a buffer; and/or
f) a tonicity adjusting agent;
said liquid nicardipine composition having a pH of at least about 4.8,
wherein said liquid nicardipine composition retains at least about 90% of its starting concentration when stored in a pharmaceutically-acceptable bag-type container under conditions of 40° C., 75% relative humidity for a period of at least about 6 months.

2. The liquid nicardipine composition of claim 1, wherein the nicardipine is nicardipine HCl.

3. The liquid nicardipine composition of claim 1, wherein the pharmaceutically acceptable bag-type container includes sufficient space therein for an amount of an inert gas to be placed therein with the nicardipine composition.

4. The liquid nicardipine composition of claim 3, wherein the inert gas is nitrogen.

5. The liquid nicardipine composition of claim 1, wherein the concentration of the nicardipine in the composition is from about 0.015 mg/mL to about 0.5 mg/mL.

6. The liquid nicardipine composition of claim 5, wherein the concentration of the nicardipine in the composition is from about 0.05 to about 0.4 mg/mL.

7. The liquid nicardipine composition of claim 6, wherein the concentration of the nicardipine in the composition is from about 0.1 to about 0.3 mg/mL.

8. The liquid nicardipine composition of claim 1, wherein the pH is in the range of about 4.8-8.0.

9. The liquid nicardipine composition of claim 8, wherein the pH is in the range of about 4.8-6.0.

10. The liquid nicardipine composition of claim 9, wherein the pH is in the range of about 5.0-5.5.

11. The liquid nicardipine composition of claim 1, wherein the hydrotropic agent is sodium benzoate.

12. The liquid nicardipine composition of claim 1, wherein the amount of the hydrotropic agent in the composition is from about 0.2 to about 2 mg/mL.

13. The liquid nicardipine composition of claim 11, wherein the amount of hydrotropic agent in the composition is from about 0.5 to about 1.5 mg/mL.

14. The liquid nicardipine composition of claim 13, wherein the amount of hydrotropic agent in the composition is about 1 mg/ml.

15. The liquid nicardipine composition of claim 1, wherein the surfactant is selected from the group consisting of tweens, spans, cremophores, phospholipids such as phosphatidyl choline, PEGylated phospholipids, 1, 2-Distearoyl-sn-glycero-3-phosphoethanolamine-Poly(ethylene glycol) (DSPE-PEG), PEGylated compounds, Cholesterol-PEG, Vitamin E-PEG and mixtures thereof.

16. The liquid nicardipine composition of claim 15, wherein the surfactant is phosphatidyl choline or 1, 2-Distearoyl-sn-glycero-3-phosphoethanolamine-Poly(ethylene glycol) (DSPE-PEG).

17. The liquid nicardipine composition of claim 1, wherein the amount of surfactant in the composition is from about 0.05 to about 5 mg/mL.

18. The liquid nicardipine composition of claim 17, wherein the amount of surfactant in the composition is from about 0.1 to about 3 mg/mL.

19. The liquid nicardipine composition of claim 18, wherein the amount of surfactant in the composition is from about 0.5 mg/mL to about 2 mg/mL.

20. The liquid nicardipine composition of claim 1, wherein the buffer comprises citric acid.

21. The liquid nicardipine composition of claim 1, wherein the amount of buffer in the composition is from about 0.05 to about 5 mg/mL.

22. The liquid nicardipine composition of claim 21, wherein the amount of buffer in the composition is from about 0.1 to about 3 mg/mL.

23. The liquid nicardipine composition of claim 22, wherein the amount of buffer in the composition is from about 0.1 to about 0.5 mg/mL.

24. The liquid nicardipine composition of claim 1, wherein the tonicity adjusting agent is selected from the group consisting of sodium chloride, glycerol, propylene glycol, dextrose, lactose, mannitol, sorbitol, sucrose, and mixtures thereof.

25. The liquid nicardipine composition of claim 24, wherein the tonicity adjusting agent is sorbitol.

26. The liquid nicardipine composition of claim 1, wherein the amount of tonicity agent in the composition is from about from about 0.1 mg/mL to about 60 mg/mL.

27. The liquid nicardipine composition of claim 26, wherein the amount of tonicity agent in the composition is from about 0.6 to about 25 mg/mL.

28. The liquid nicardipine composition of claim 27, wherein the amount of tonicity agent in the composition is from about 0.8 to about 10 mg/mL.

29. The liquid nicardipine composition of claim 1, wherein the nicardipine retains at least about 95% of its starting concentration when stored in a pharmaceutically-acceptable bag-type container under conditions of 40° C., 75% relative humidity for a period of at least about 6 months.

30. A pharmaceutically acceptable container comprising a therapeutically acceptable amount of a liquid nicardipine composition of claim 1, wherein the pharmaceutically-acceptable container is a pharmaceutically-acceptable plastic bag-type container.
